(19) United States
(12) Patent Application Publication
(10) Pub. No.: US 2012/0315281 A1
(43) Pub. Date: Dec. 13, 2012

(54) TREATMENT OF RENAL INJURY

(75) Inventor: Alaa S. AWAD, Hershey, PA (US)

(73) Assignee: THE PENN STATE RESEARCH FOUNDATION, University Park, PA (US)

(21) Appl. No.: 13/469,731

(22) Filed: May 11, 2012

Related U.S. Application Data
(60) Provisional application No. 61/485,671, filed on May 13, 2011.

(51) Int. Cl. A61K 31/198; A61K 39/395; G01N 33/573; C40B 30/08; C12Q 1/34; C12Q 1/68; A61K 31/711; A61P 13/12

(52) U.S. Cl. 424/158.1; 514/564; 514/44 R; 506/11; 435/18; 435/6.12; 435/7.4

(57) ABSTRACT

Methods for the treatment of renal injury include the use of arginase inhibitors in the treatment and prevention of diabetic nephropathy, albuminuria and azotemia amongst others, and methods for identifying agents for the treatment of renal injury.

### CROSS-REFERENCE TO RELATED APPLICATIONS

The application is a continuation-in-part, which claims the priority of U.S. provisional patent application No. 61/485,671 entitled “TREATMENT OF RENAL INJURY”, filed May 13, 2011, which is incorporated herein by reference in their entirety.

### STATEMENT REGARDING FEDERALLY SPONSORED RESEARCH

This invention was made with U.S. government support under grant numbers DK077444 awarded by the National Institutes of Health. The U.S. government may have certain rights in the invention.

### FIELD OF THE INVENTION

Embodiments of the invention comprise methods of treatment and prevention of renal injury, including diabetic nephropathy.

### BACKGROUND

Diabetes is a global health problem. In the United States, 25.8 million children and adults have diabetes, with at least 1.9 million new cases of diabetes diagnosed in 2010. American Diabetes Association, 2011 National Diabetes Fact Sheet. Diabetic nephropathy has become one of the principal causes of end-stage renal disease, leading to dialysis and death in the Western population. S. Gray & M. Cooper, Nature Reviews Nephrology 7, 71-73 (February 2011). Over the past decade, the incidence of end-stage renal disease due to diabetes has doubled, and the incidence is likely to continue to increase unabated. Current therapies include blood pressure and glucose control and other lifestyle changes and have only been modestly successful in delaying the progression of renal failure.

Dramatic alterations in arginine metabolism correlate strongly with the progression of tissue injury, including vascular dysfunction in diabetes. However, the direct role of arginases in the progression of diabetic nephropathy remains unknown. In most mammals, two isoenzymes of arginase exist (arginase-1 and arginase-2). Arginase-2 is the predominant isoform normally expressed in kidney. Arginase catalyzes the reaction of arginine+H₂O→ornithine+urea in the final step of the urea cycle. Depending on stimulus, either one or both of the arginases may be expressed and induced in macrophages, endothelial cells, and other cell types.

### SUMMARY

This Summary is provided to present a summary of the invention to briefly indicate the nature and substance of the invention. It is submitted with the understanding that it will not be used to interpret or limit the scope or meaning of the claims.

Embodiments of the invention are directed to methods of treating renal injury including administering to a subject in need thereof, a therapeutically effective amount of an arginase inhibitor and treating the subject's renal injury. In some embodiments, the therapeutically effective amount of the arginase inhibitor can be administered together with a pharmaceutically acceptable carrier. In some embodiments, the therapeutically effective amount of the arginase inhibitor can be administered intravenously. In some embodiments, the therapeutically effective amount of the arginase inhibitor can be administered through intraperitoneal injection. In some embodiments, the therapeutically effective amount of the arginase inhibitor can be administered orally. In another embodiment, the subject to whom the therapeutically effective amount of an arginase inhibitor is administered is a human.

Other aspects are described infra.

### BRIEF DESCRIPTION OF THE DRAWINGS

FIG. 1 shows the effect of arginase inhibition on urinary albumin excretion (UAE) in Ins2^{Akita} mice. The Ins2^{Akita} and their wild type (WT) littermate mice were treated with the arginase-specific inhibitor S-(2-Boronoethyl)-L-cysteine (BEC) (2.3 mg/kg/day) or vehicle via osmotic minipump for 9 weeks. Urine was collected for measurement of UAE before treatment (at 5 weeks of age) and after treatment (at 14 weeks of age). In FIG. 1, the open bars are vehicle treated groups and filled bars are arginase inhibitor treated groups. Data are presented as mean±SEM.

FIGS. 2A-2D show the effect of arginase inhibition on macrophage recruitment in Ins2^{Akita} mice using immunohistochemistry. FIG. 2A shows immunohistochemical staining for Mac-2 positive macrophages in glomeruli at 14 weeks of age in control mice treated with vehicle; FIG. 2B shows immunohistochemical staining for Mac-2 positive macrophages in glomeruli at 14 weeks of age in Ins2^{Akita} mice treated with vehicle; FIG. 2C shows immunohistochemical staining for Mac-2 positive macrophages in glomeruli at 14 weeks of age in control mice treated with arginase inhibitor; and FIG. 2D shows immunohistochemical staining for Mac-2 positive macrophages in glomeruli at 14 weeks of age in Ins2^{Akita} mice treated with arginase inhibitor.

FIG. 3 shows the effect of arginase inhibition on macrophage recruitment in Ins2^{Akita} mice using flow cytometry (FACS). Kidneys were harvested at 14 weeks of age, processed for FACS, and macrophages were identified as CD11b^{high}F4/80^{low}. The graphs in FIG. 3 show representative contour plots.

FIG. 4 shows kidney mRNA expression of arginase 1 and arginase 2.

FIGS. 5A and 5B show the effect of arginase inhibition on arginase 2 mRNA expression in Ins2^{Akita} mice. Real-time polymerase chain reaction (RT-PCR) was performed on whole mouse kidney total RNA isolated at 14 weeks of age. FIG. 5A shows a gel analysis of PCR products. In FIG. 5B, expression of arginase 2 mRNA was normalized to GAPDH and data were calculated as expression relative to control.

FIG. 6 shows the effects of arginase inhibition on UAE in STZ-induced diabetic mice (DBA background). After an overnight fast, animals were given multiple low doses of vehicle or STZ (Sigma, St. Louis, Mo.; 50 mg/kg body wt dissolved in lactated Ringers solution) via IP injection. Mice were treated with the arginase-specific inhibitor BEC (2.3 mg/kg/day) or vehicle via osmotic minipump for 6 weeks. Urine was collected for measurement of UAE after 6 weeks. Data are presented as mean±SEM. FIG. 6 shows *p<0.05, **p<0.0001 compared to normal, #p<0.05 compared to diabetes+vehicle group.

FIGS. 7A-7C shows the effects of arginase inhibition on histological changes in STZ-induced diabetic mice (DBA background). Sections were stained with Periodic acid-Schiff (PAS) and all glomeruli were examined at 40×. Images were taken with 100× (oil) objective with a total magnification of 1000×. FIG. 7A shows a representative PAS section from the normal group showing morphologically normal glomerulus with delicate PAS positive basement membranes and minimal PAS staining of mesangial matrix. Adjacent tubular basement membranes stain PAS positive as well. FIG. 7B shows a representative PAS section from a vehicle treated STZ mouse showing glomerular expansions in which PAS positive material occupies 25-50% of the mesangial matrix within the tuft. FIG. 7C shows a representative PAS section from an arginase inhibitor treated STZ mouse showing glomerular expansion in which PAS positive material occupies <25% of the mesangial matrix.

FIG. 8 shows the effect of arginase 2 deletion on UAE in diabetes. Urine was collected for measurement of UAE in arginase 2^{+/+} (WT mice) and arginase 2^{-/-} (KO mice) at baseline, week 6 and week 18 of the study to determine if arginase 2 contributes to diabetic renal injury. The open bars are WT mice and the filled bars are arginase 2 KO mice.

FIG. 9 shows the effect of arginase 2 deletion on renal medullary blood flow in diabetic mice. Renal medullary blood flow was measured after 6 weeks of STZ-induced diabetes using a Transonic flow probe (Transonic System Inc., Ithaca, N.Y.). The open bars are the control group and the filled bars are the diabetes group.

FIG. 10 shows the effect of STZ-induced diabetes on kidney arginase activity. Kidney lysates were prepared using lysis buffer (50 mM Tris-HCl, pH7.5, 0.1 mM EDTA and protease inhibitors) by homogenization at 4° C. followed by centrifugation for 20 min at 14,000×g at 4° C. The supernatants were used to assay for arginase activity. The open bars are WT mice and the filled bars are arginase 2 KO mice.

FIGS. 11A-11C show arginase-2 expression in mouse kidney-derived cells. Experiments were conducted with murine glomerular endothelial cells. These cells were differentiated for 10 days at 37° C. in DMEM/F12 supplemented with either normal glucose (NG) (11 mM) or HG (33 mM). HG media increased the arginase-2 mRNA level in glomerular endothelial cells by 158±15% (p<0.005) compared to NG (FIG. 11A). Arginase-2 expression was confirmed in podocytes (FIG. 11B) and isolated glomeruli (FIG. 11C).

FIG. 12 shows immunohistochemical staining for arginase-2 on representative kidney sections. The images are shown 1000× oil immersion (original and cropped). Arginase-2 was expressed in proximal straight tubules and mesangial cells. Furthermore, both Tie2hArg2 transgenic and WT mice show strong granular cytoplasmic (mitochondrial) staining for arginase-2 in tubular epithelial cells, with specific staining within endothelial cells, mesangial cells, and visceral podocytes. Arg2^{-/-} mouse had no specific staining, confirming specificity of our immunohistochemistry. These data provide evidence for a possible role for arginase-2 in these kidney cell types.

FIG. 13A shows RT-PCR performed on unidentified kidney samples from diabetic patients (n=3). B-actin was used as a control gene.

FIG. 13B shows immunohistochemical staining for arginase-2 in representative kidney sections from diabetic human samples. The image shows granular cytoplasmic (mitochondrial) staining in tubular epithelial cells, endothelial, mesangial cells and podocytes. The images are shown 1000× oil immersion (original and cropped).

FIG. 14 shows the effects of arginase inhibition on renal function in male 6-week-old eNOS deficient (eNOS KO) and their wild type (WT) littermate mice. Experiments were conducted using multiple low doses of STZ (50 mg/kg body weight for 5 days). Arginase inhibition using BEC (2.3 mg/kg/day) or vehicle was administered by continuous subcutaneous infusion for 6 weeks via a mini-osmotic pump. Data is shown for 6 weeks after STZ-induced diabetes.

FIG. 15 shows a schematic representation of the study protocol used.

### DETAILED DESCRIPTION

Embodiments of the invention relate to discoveries involving methods for the treatment and prevention of renal injury including the use of arginase inhibitors, and methods for identifying agents for the treatment of renal injury.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a,” “an” and “the” are intended to include the plural forms as well, unless the context clearly indicates otherwise. Furthermore, to the extent that the terms “including,” “includes,” “having,” “has,” “with” or variants thereof are used in either the detailed description and/or the claims, such terms are intended to be inclusive in a manner similar to the term “comprising.”

“Treating” or “treatment” of an injury, state, disorder or condition includes: (1) preventing or delaying the appearance of clinical or sub-clinical symptoms of the injury, state, disorder or condition developing in a mammal that may be afflicted with or predisposed to the injury, state, disorder or condition but does not yet experience or display clinical or subclinical symptoms of the injury, state, disorder or condition; or (2) inhibiting the injury, state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the injury, state, disorder or condition or at least one of its clinical or sub-clinical symptoms. The benefit to a subject to be treated is either statistically significant or at least perceptible to the patient or to the physician.

As used herein, “renal injury” refers to any renal disease; any other injury, state, disorder or condition adversely affecting kidney function; and any state, disorder, condition or symptoms associated with renal disease. For example, renal injury may include nephropathy, diabetic nephropathy, glomerulopathy, diabetic kidney disease, glomerular injury, nephritis, hyperfiltration, kidney failure, and associated conditions such as proteinuria including albuminuria; azotemia; and increased kidney macrophage recruitment.

The terms “patient” or “subject” are used interchangeably herein, and refer to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein “a subject in need thereof” refers to any subject that is affected with renal injury. In one aspect of the invention “a subject in need thereof” refers to any subject that may have, or is at risk of developing renal injury.

As defined herein, a “therapeutically effective” amount of an agent (i.e., an effective dosage) means an amount sufficient to produce a therapeutically (e.g., clinically) desirable result. Desirable results may include, without limitation, preventing, alleviating or relieving the symptoms of an injury, illness or disease. The agents can be administered from one or more times per day to one or more times per week; including once every other day. The skilled artisan will appreciate that certain factors can influence the dosage and timing required to effectively treat a subject, including but not limited to the severity of the injury, disease or disorder, previous treatments, the general health and/or age of the subject, and other diseases present. Moreover, treatment of a subject with a therapeutically effective amount of the agents according to the invention can include a single treatment or a series of treatments.

As used herein, an “arginase inhibitor” refers to any agent capable of inhibiting the function, expression, activity or combinations thereof, of the arginase-2 enzyme and/or the arginase-2 gene (ARG2), the arginase 1 enzyme and/or the arginase-1 gene (ARG1) or both the arginase 1 and 2 enzymes and/or the arginase 1 and 2 gene; and/or any agent capable of increasing levels of arginase's substrate, arginine.

The terms “arginase,” “arginase-1” and “arginase-2,” are inclusive of all species, including human.

As used herein, “inhibition” and “inhibiting” refers to a decrease in the effect, function, activity or combinations thereof effect of a target from the normal effect, function or activity produced by or otherwise attributed to the target.

As used herein, the phrases “selective inhibition” and “selectively inhibiting” refer to inhibiting the function, expression or activity of a target to a greater degree in comparison to similar or analogous compounds. For example, an agent may selectively inhibit arginase 2 activity or expression, selectively inhibit arginase 1 activity or expression, or selectively inhibit both arginase 1 and 2 activity or expression.

As used herein, a “pharmaceutically acceptable” carrier is one that is suitable for use with humans and/or animals without undue adverse side effects (such as toxicity, irritation, and allergic response) commensurate with a reasonable benefit/risk ratio.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the terms “oligonucleotide,” “siRNA,” and “antisense oligonucleotide” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

The terms “test substance” or “agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The terms include small molecule compounds, antisense reagents, siRNA reagents, antibodies, enzymes, peptides organic or inorganic molecules, natural or synthetic compounds and the like.

### Treatment of Renal Injury and Inhibition of Arginase

Arginase is an enzyme that catalyzes the conversion of arginine to ornithine and urea in the urea cycle. Specifically, arginase is a binuclear manganese metalloenzyme which hydrolyzes L-arginine through a metal-activated hydroxide mechanism. The binuclear manganese clusters are required for proper orientation and stabilization of L-arginine for hydrolysis. In a different metabolic pathway, the enzyme nitric oxide synthase catalyzes the conversion of L-arginine to nitric oxide and citrulline. Thus, arginase competes with nitric oxide synthase for the L-arginine substrate. In some embodiments, the arginase inhibitor increases nitric oxide production, action or function, bioavailability or activity.

There are two isoforms of arginase, identified as arginase-1 and arginase-2. Although arginase-1 and arginase-2 have similar enzyme activities, they differ in tissue distribution, subcellular localization, isoelectric points (pI) and immunological reactivity, they are encoded by different genes—ARG1 and ARG2, and they are independently regulated. In humans, ARG1 maps to chromosome 6q23, while ARG2 maps to chromosome 14q24.1-q24.3. Arginase-2 is located in the mitochondria and expressed in extra-hepatic tissues, including the kidney. In addition to arginine/ornithine regulation, arginase-2 may also be involved in nitric oxide and polyamine metabolism.

Briefly, the data described in detail in the Examples section which follows, show that pharmacological blockade of arginase-1 or arginase-2 or both arginase-1 and -2 or genetic deficiency of arginase-2 confers kidney protection in Ins2^{Akita} or streptozotocin (STZ)-induced diabetic kidney disease. Such kidney protection may include attenuated albuminuria, blood urea nitrogen levels, histological changes and kidney macrophage recruitment.

In a preferred embodiment, a method for the treatment of renal injury comprises the steps of administering to a subject in need thereof, a therapeutically effective amount of an arginase inhibitor and/or an agent which modulates arginine expression, levels, or function and treating renal injury. Examples of the arginase inhibitor are further discussed below.

The method can also include administering the therapeutically effective amount of the arginase inhibitor together with a pharmaceutically acceptable carrier. The pharmaceutically acceptable carrier can include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the compositions.

The method can also include administering the therapeutically effective amount of the arginase inhibitor though various delivery mechanisms. For example, administering the therapeutically effective amount of the arginase inhibitor can include, without limitation, through intraperitoneal (IP) injection, intravenous (IV) injection, subcutaneous (SC) injection, oral, lipid microsphere (LM) preparations, topical administration and oral administration or mixed with food. Administering the arginase inhibitor can also include various delivery forms, for example liquids, capsules, pills, powders, granules, elixirs, tinctures, suspensions, syrups, food and emulsions.

In another preferred embodiment, the subject can be a human.

In another preferred embodiment, the renal injury can include diabetic nephropathy. In other embodiments, the diabetic renal injury can include hyperfiltration, albuminuria, azotemia, nephritis, and kidney failure.

In another preferred embodiment, a method for inhibiting arginase in vivo comprises the steps of administering in vivo to a subject in need thereof, a therapeutically effective amount of an arginase inhibitor such that inhibition of arginase treats renal injury.

The arginase inhibitor can include any agent capable of inhibiting the function, expression, activity or combinations thereof, of arginase-2 and/or ARG2, arginase-1 and/or ARG1, or both arginase-1 and -2 and/or ARG1 and ARG2. In another preferred embodiment, the arginase inhibitor can also selectively inhibit arginase-2 or arginase-1 or both. For example, the arginase inhibitor can include competitive inhibitors that bind to the active site of arginase-1 or arginase-2 or both. The arginase inhibitor can include a boronic-acid based agent, such as BEC. The arginase inhibitor can also include an oligonucleotide specific for ARG2 or ARG1 or both, including siRNAs and antisense oligonucleotides. The arginase inhibitor can also include an antibody specific for arginase-2 enzymes, arginase-1 enzymes or both arginase-1 and -2 enzymes. The arginase inhibitor can also include an L-arginine supplement. As used herein, “L-arginine supplement” refers to any agent capable of increasing the presence of L-arginine in a subject compared to the level of L-arginine present in the absence of the L-arginine supplement. The L-arginine supplement can also include natural or synthetic L-arginine, variants, analogs, derivatives, enantiomers, substitutions, fragments or combinations thereof.

As used herein, “L-citrulline supplement” refers to any agent capable of increasing the presence of L-citrulline in a subject compared to the level of L-citrulline present in the absence of the L-citrulline supplement. The L-citrulline supplement can also include natural or synthetic L-citrulline, variants, analogs, derivatives, enantiomers, substitutions, fragments or combinations thereof.

L-citrulline, is a precursor of arginine. Just as arginine is converted to citrulline and NO, L-citrulline is converted to arginine in the mitochondria. The majority of circulating L-citrulline is converted in the kidneys, which are comprised of highly metabolically active tissue. As such, L-citrulline circulating in the bloodstream is first converted to arginine and then in cells to citrulline and NO. Significantly, the conversion of L-citrulline to arginine occurs continuously, as long as L-citrulline is circulating in the bloodstream. As a result, circulating L-citrulline makes it possible to maintain elevated concentrations of arginine over time, which in turn makes it possible to maintain a steady release of NO in cells

The methods of modulating arginase and/or arginine and/or L-citrulline described herein can also treat one or more diseases or disorders associated with abnormal arginase expression or levels in a patient as compared to healthy normal controls. Diseases or disorders associated with abnormal arginase expression or levels in a patient as compared to healthy normal controls can include renal diseases or disorders, renal injury, diabetic nephropathy, angina, congestive heart failure, cancer, azotemia, albuminuria, nephritis, renal failure or cardiovascular diseases.

Examples of an arginase inhibitor comprises: 2(S)-amino-6-boronohexanoic acid (ABH), S-(2-boronoethyl)-L-cysteine (BEC), NΩ-OH-L-arginine (NOHA), NΩ-hydroxy-nor-L-arginine (nor-NOHA), α-difluoromethylornithine (DFMO), L-norvaline, iodoacetyl-L-ornithine, iodoacetyl-L-lysine, L-lysine or combinations thereof. Included are the pharmaceutically acceptable salts of these compounds. These examples are not meant to be limiting.

### Identifying Agents for Modulating Arginases

A further aspect of the present invention relates to methods of identifying agents or candidate agents for modulating arginase and/or arginine and/or L-citrulline suitable for treating of renal injury. The term “arginase inhibitors” or “modulators” will be used interchangeably and the terms are for illustrative purposes but it is to be understood that it includes any agent which modulate arginases, arginine or L-citrulline, or combinations thereof. This includes the modulation of function, activity, expression, bioavailability and the like. The agents can be used alone or in combinations with other therapeutic agents to treat patients.

In a preferred embodiment, a method for identifying an agent for modulating arginase in vitro or in vivo comprises the steps of providing a test substance, determining whether the test substance is an arginase inhibitor, and selecting the test substance for treatment of renal injury if the test substance is an arginase inhibitor. The method can also include the step of determining whether the test substance selectively inhibits arginase and selecting the test substance for treatment of diabetic renal injury if the test substance is an arginase inhibitor and selectively inhibits arginase. A similar assay can also be employed for identifying agents which modulate L-arginine expression, or L-citrulline expression, function and/or levels in vitro or in vivo.

Determining whether a test substance selectively inhibits arginase can be conducted with any suitable method including those well known in the art. For example, the expression of ARG2 can be determined using RT-PCR or Western Blot analysis and comparing the expressions of ARG2 in subjects administered the test substance and control subjects no administered the test substance.

In a preferred embodiment, methods (also referred to herein as “screening assays”) are provided for identifying modulators, i.e., candidate or test compounds or agents (e.g., proteins, peptides, peptidomimetics, peptoids, small molecules, analogues or other drugs) which modulate which modulate arginases, arginine or L-citrulline, or combinations thereof, of for example, their activity, function, expression, bioavailability or synthesis pathways thereof. Compounds thus identified can be used to modulate the activity of target gene products, prolong the half-life of a protein or peptide, regulate cell division, etc., in a therapeutic protocol, to elaborate the biological function of the target gene product, or to identify compounds that disrupt normal target gene interactions.

In another preferred embodiment, a high-throughput screening assay (HTS) screening assay is used to screen a diverse library of member compounds. The “compounds” or “candidate therapeutic agents” or “candidate agents” or “arginase inhibitors” or “modulators” can be any organic, inorganic, small molecule, protein, antibody, aptamer, nucleic acid molecule, or synthetic compound.

In another preferred embodiment, the candidate agents modulate the arginase enzymes, precursors or molecules involved in the pathways. Preferably, the enzyme is arginase 1 or arginase 2.

In another preferred embodiment, the assay is a high throughput assay.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Other examples of candidate agents comprise: amino acids, nucleic acids, oligonucleotides, polynucleotides, peptide nucleic acids, peptides, polypeptides, antibodies, small molecules, organic or inorganic molecules, synthetic molecules, natural molecules, variants, analogs, or combinations thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the one-bead one-compound library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carrell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Nat'l Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In another preferred embodiment, the candidate therapeutic agent comprises proteins, peptides, organic molecules, inorganic molecules, nucleic acid molecules, and the like. These molecules can be natural, e.g. from plants, fungus, bacteria etc., or can be synthesized or synthetic.

A prototype compound may be believed to have therapeutic activity on the basis of any information available to the artisan. For example, a prototype compound may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC₅₀, assay data, IC₅₀ assay data, animal or clinical studies, or any other basis, or combination of such bases.

A therapeutically-active compound is a compound that has therapeutic activity, including for example, the ability of a compound to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against any disease or disorder associated with renal injury. In order to determine therapeutic activity any method by which therapeutic activity of a compound may be evaluated can be used. For example, both in vivo and in vitro methods can be used, including for example, clinical evaluation, EC₅₀, and IC₅₀ assays, and dose response curves.

Candidate compounds for use with an assay of the present invention or identified by assays of the present invention as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomer, or small molecule libraries of compounds and are preferred approaches in the present invention. See Lam, Anticancer Drug Des. 12: 145-167 (1997).

In an embodiment, the present invention provides a method of identifying a candidate compound as a suitable prodrug. A suitable prodrug includes any prodrug that may be identified by the methods of the present invention. Any method apparent to the artisan may be used to identify a candidate compound as a suitable prodrug.

In another aspect, the present invention provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, bioavailability, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

A further aspect of the present invention relates to methods of inhibiting the activity of a condition or disease associated with renal injury comprising the step of treating a sample or subject believed to have a disease or condition with a prodrug identified by a compound of the invention. Compositions of the invention act as identifiers for prodrugs that have therapeutic activity against a disease or condition. In a preferred aspect, compositions of the invention act as identifiers for drugs that show therapeutic activity against conditions including for example associated with renal injury, e.g. diabetes etc.

In one embodiment, a screening assay is a cell-based assay in which the activity of arginase inhibitor is measured against an increase or decrease of NO, for example. Determining the ability of the test compound to modulate the ariginases' functions, activity etc, can be done as discussed herein, by various methods, including for example, fluorescence, protein assays, blots and the like. The cell, for example, can be of mammalian origin, e.g., human.

Cell-free assays can also be used and involve preparing a reaction mixture which includes arginases and the test compound under conditions and time periods to allow the measurement of the arginase activity, for example, over time, concentrations of test agents etc.

In one embodiment, the target product or the test substance is anchored onto a solid phase. The target product/test compound complexes anchored on the solid phase can be detected at the end of the reaction. Preferably, the target product can be anchored onto a solid surface, and the test compound, (which is not anchored), can be labeled, either directly or indirectly, with detectable labels discussed herein.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries

Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol. Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids; encoded peptides; random bio-oligomers; benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

Small Molecules

Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

The whole procedure can be fully automated. For example, sampling of sample materials may be accomplished with a plurality of steps, which include withdrawing a sample from a sample container and delivering at least a portion of the withdrawn sample to test platform. Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting examples serve to illustrate selected embodiments of the invention. It will be appreciated that variations in proportions and alternatives in elements of the components shown will be apparent to those skilled in the art and are within the scope of embodiments of the present invention.

### Materials and Methods

Diabetic Mouse Models

Experiments were conducted in male Ins2^{Akita} and their wild type (WT) littermate mice (DBA background; Jackson Laboratories) starting at 5 weeks of age until 14 weeks of age and were approved by the Pennsylvania State University College of Medicine Institutional Animal Care and Use Committee (PSU IACUC). The Ins2^{Akita} mice were recommended by the Animal Models of Diabetes Complications Consortium as an optimal model of diabetic nephropathy, and the mice developed hyperglycemia at 3 weeks of age. Additional experiments were conducted in male 6-week-old mice (DBA background; Jackson Laboratories), arginase-2 (Arg2) wild type (Arg2^{-/-}) and deficient (Arg2^{+/+}) mice on C57B1/6 background weighing 20-22 g and were approved by the PSU IACUC. After an overnight fast, animals were given multiple low doses of vehicle or STZ (Sigma, St. Louis, Mo.; 50 mg/kg body wt dissolved in lactated Ringers solution) via intraperitoneal injection.

The study protocol used is shown in FIG. 15.

Drug Delivery

BEC, a selective arginase antagonist (2.3 mg/kg/day, Cayman Chemical, Ann Arbor, Mich., USA), or vehicle (phosphate-buffered saline) was administered by continuous subcutaneous infusion for 9 weeks (in Ins2^{Akita} experiments) or for 6 wks (in STZ DBA mice experiments) via a mini-osmotic pump (Alzet; Durect Corporation, Palo Alto, Calif., USA) as described in CIRCULATION RESEARCH, 102(8):923-32, Apr. 25, 2008. The osmotic pump was incubated in phosphate buffer saline (PBS) for 60-h at 37° C. prior to implantation and was implanted dorsally between shoulders. The condition of mice and body weight were monitored daily following the pump implantation.

Blood Pressure Measurement

Systolic blood pressure was measured using Coda blood pressure (Kent Scientific Corp, Torrington, Conn.). Mice were allowed to rest quietly for 10 minutes at 26° C. All measurements were performed at the same time for all groups to prevent any diurnal variations.

Renal Histochemistry and Immunohistochemistry

Kidneys from mice were fixed in 4% paraformaldehyde, embedded in paraffin, and 3-μm sections were cut. Sections were stained with Periodic acid-Schiff (PAS) stain and all glomeruli were examined at 40× (TKC) in a masked fashion. All images were obtained with an Olympus BX51 microscope and DP71 digital camera using MicroSuite Basic 2.6 imaging software. Images were taken with 100× (oil) objective with a total magnification of 1000×. Semiquantitative scores (0-4+) were assigned based on the masked reading, as previously described. Each glomerulus on a single section was graded from 0 to 4+, where 0 represents no lesion, and 1, 2, 3, and 4+ represent mesangial matrix expansion or sclerosis, involving ≦25 to 50, 50 to 75, or >75% of the glomerular tuft area, respectively.

Immunohistochemistry for macrophages was performed in mice using rat anti-mouse Mac-2 monoclonal antibody; clone M3/38 (Cedarlane, Burlington, N.C., US) on paraffin sections. Sections were incubated with primary antibody (50 μg/ml) followed by a biotinylated goat IgG anti-rat (Vector Laboratories, Burlingame, Calif.) secondary antibody according to the manufacturer's protocol. Sections were viewed using an Olympus BX51 microscope and DP71 digital camera using MicroSuite Basic 2.6 imaging software. Images were taken with 100× (oil) objective with a total magnification of 1000×. The number of glomerular macrophages were counted in twenty glomeruli per section (number of macrophages in glomeruli divided by the number of glomeruli) in blinded fashion under 40× magnification and averaged.

Analysis of Kidney Macrophage Content by FACS

Flow cytometry (FACS) was used to analyze kidney macrophage content (CD11b⁺F4/80^{low}) at the end of experiments as described previously. In brief, kidneys were extracted, minced, digested, and then passed through a filter and a cotton wool column. Fresh kidney suspensions were incubated with anti-mouse CD45-FITC (30-F11; eBioscience, San Diego, Calif.) for 30 minutes on ice. Kidney macrophages were then identified using allophycocyanin (APC)-labeled rat anti-mouse F4/80 (BM8; eBioscience) and phycoerythrin (PE)-labeled rat anti-mouse CD11b (M1/70; eBioscience). All samples were treated with anti-mouse CD16/CD32 (2.4G2) to block the nonspecific FcR binding and 7-AAD to eliminate dead cells (Invitrogen, Carlsbad, Calif.). Counting beads (Caltag, Carlsbad, Calif.) were used to determine the total number of CD45⁺ cells per gram of kidney tissue. Subsequent flow cytometry data acquisition was performed on FASCalihur (Becton Dickinson, San Jose, Calif.). Data were analyzed by Flowjo software 6.4 (Tree Star, Ashland, Oreg.). All of the antibodies were purchased from eBioscience (San Diego, Calif.).

Quantitative Real-Time PCR

Total RNA was extracted from mouse kidneys using RNeasy Mini Kit (Qiagen, Gumbh, Hilden, Germany). Single-strand cDNA was synthesized using iScript cDNA Synthesis Kits (Bio-Rad, Hercules, Calif.) for two-step real-time polymerase chain reaction (RT-PCR). Gene-specific primers for ARG1 and ARG2 were designed using Beacon Designer Probe/Primer Design Software (Premier Biosoft International, Palo Alto, Calif.). Specificity of the PCR products was verified by melting curve analysis. Quantitative real-time PCR was performed using a Bio-Rad CFX96 system (Hercules, Calif.). Reactions were performed in duplicate, and threshold cycle numbers were averaged. Samples were calculated with normalization to GAPDH.

Analytical Methods

Urinary albumin excretion (UAE) was measured by ELISA using Albuwell M (Exocell, Philadelphia, Pa.) as described previously. Blood urea nitrogen (BUN) was measured using (VITROS DT60II chemistry slides; Ortho-Clinical Diagnostics, Rochester, N.Y.). Body composition was determined using LF90 Minispec Time Domain Nuclear Magnetic Resonance Spectrometer (Bruker Optics, Billerica Mass.).

Arginase Activity Assay

Tissue lysates were prepared using lysis buffer (50 mM Tris-HCl, pH7.5, 0.1 mM EDTA and protease inhibitors) by homogenization at 4° C. followed by centrifugation for 20 min at 14,000×g at 4° C. The supernatants were used to assay for arginase activity as described in D. Kepka-Lenhart, D. E. Ash, S. M. Morris, Determination of mammalian arginase activity, METHODS ENZYMOLOGY 440:221-230 (2008).

Statistical Analysis

Comparisons between groups were examined by using the SPSS version 19.0 software for Windows (SPSS, Chicago, Ill.) program. Data are expressed as mean±SEM. One-way ANOVA were used when more than two groups were compared and the significance of observed differences among the groups was evaluated with a least significant difference post hoc test. Statistical significance was identified at p<0.05.

### Results

Characteristics of Ins2^{Akita} Experiment

To assess the possible clinical significance of arginases in diabetic mice, the arginase-specific inhibitor BEC (2.3 mg/kg/day) was infused continuously as previously described or vehicle into Ins2^{Akita} mice and their wild-type littermates (DBA-background) for 9 weeks beginning at 5 weeks of age. As shown in Table 1, Ins2^{Akita} vehicle-treated mice showed increased blood glucose (BG) levels, decreased body weight (BW), increased kidney weight, increased kidney weight to BW ratio, reduced fat and fluid composition, and increased lean composition compared to control mice. Arginase inhibition of Ins2^{Akita} mice significantly reduced kidney weight, and kidney weight to BW ratio; without affecting other measurements. There were no significant changes in systolic blood pressure (SBP) between all groups although there was a trend toward increased SBP in the Ins2^{Akita} groups.

TABLE 1
Characteristics of Ins2^{Akita} and their wild type littermate mice (with or
without arginase inhibitor; n = 6-8 each group) at 14-week of age:

| | Control | | Ins2^{Akita} | |
|---|---|---|---|---|
| | | Arginase | | Arginase |
| | Vehicle | inhibitor | Vehicle | inhibitor |
| Glucose (mg/dl) | 167 ± 8 | 142 ± 7 | 466 ± 15*** | 441 ± 17⁺⁺⁺ |
| Body Weight (g) | 33 ± 2 | 32 ± 1 | 27 ± 1** | 27 ± 1⁺⁺ |
| Kidney weight, mg | 271 ± 7 | 262 ± 4 | 322 ± 6*** | 269 ± 10^{####} |
| KW/BW, mg/g | 8.4 ± 0.4 | 8.1 ± 0.1 | 12 ± 0.3*** | 1 0 ± 0.3++^{##} |
| BUN | 22 ± 2 | 28 ± 3 | 47 ± 8* | 28 ± 3# |
| Systolic BP (mmHg) | 112 ± 7 | 118 ± 10 | 127 ± 1 | 134 ± 2 |
| Fat; % | 13.3 ± 0.9 | 12.4 ± 0.9 | 6.8 ± 0.3*** | 6.7 ± 0.6⁺⁺⁺ |
| Lean; % | 66.7 ± 1.1 | 66.3 ± 0.5 | 70.7 ± 0.8** | 67.6 ± 1.2 |
| Fluid; % | 7.9 ± 0.2 | 7.9 ± 0.1 | 7.1 ± 0.4 | 6.4 ± 0.1⁺ |

Data in Table 1 are mean ± SEM.
*p < 0.005,
**p < 0.001,
***p < 0.0001 to control + vehicle group;
⁺p < 0.01,
⁺⁺p < 0.005,
⁺⁺⁺p < 0.0001 to control + arginase antagonist group;
#p < 0.05,
^{##}p < 0.001,
^{###}p < 0.0001 to Ins2^{Akita} + vehicle group.
KW/BW: kidney weight to body weight ratio.

Inhibition of Arginases Reduces Albuminuria and Blood Urea Nitrogen in Ins2^{Akita} Mice

To determine if arginases contribute to diabetic renal injury, the Ins2^{Akita} and their wild type littermate mice were treated with the arginase-specific inhibitor BEC (2.3 mg/kg/day) or vehicle via osmotic minipump for 9 weeks. 24-hr UAE and blood urea nitrogen (BUN) were measured before treatment at 5 weeks of age and after treatment at 14 weeks of age as indicators of renal injury in Ins2^{Akita} with and without BEC treatment.

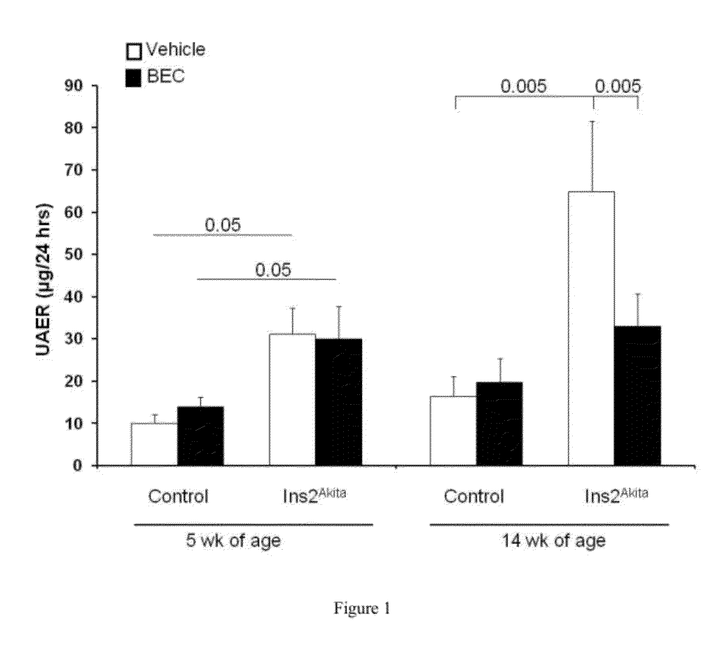

As shown in FIG. 1, vehicle-treated Ins2^{Akita} mice had a significant increase in albuminuria compared to controls at 5 and 14 weeks of age. Albuminuria was significantly reduced in Ins2^{Akita} mice treated with BEC at 14 weeks of age. Similarly, BUN was significantly increased in vehicle-treated Ins2^{Akita} mice compared to other groups (see Table 1).

Inhibition of Arginases Decreases Macrophage Recruitment in Ins2^{Akita} Mice

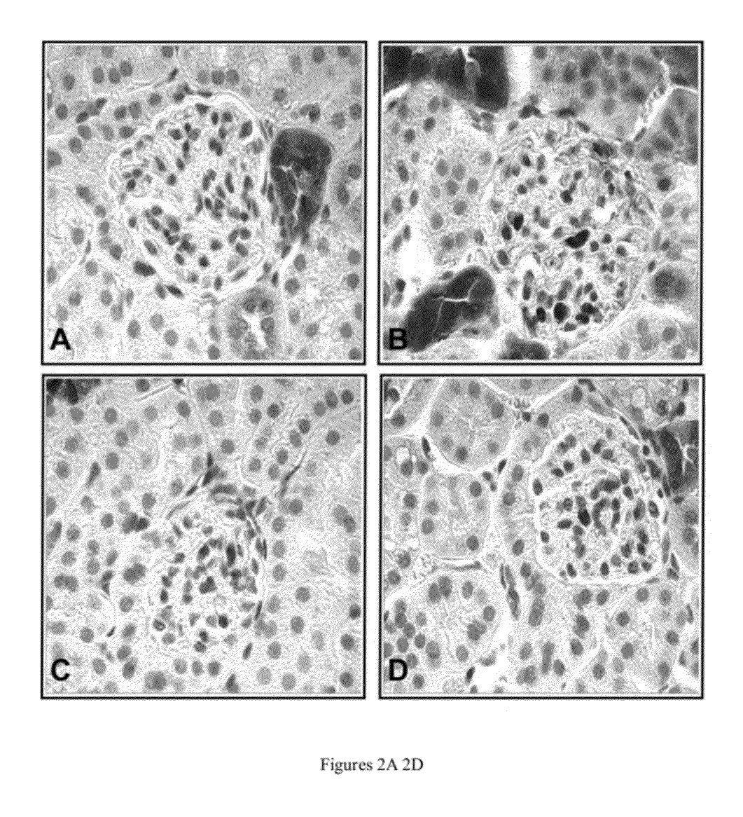

To determine whether arginases are critical for kidney macrophage infiltration in diabetic nephropathy, the distribution and quantitation of macrophages in kidneys by immunohistochemistry (Mac-2 positive macrophages) were analyzed. See FIGS. 2A to 2D. The figures show immunohistochemical staining for Mac-2 positive macrophages in glomeruli at 14 weeks of age in control mice treated with vehicle (FIG. 2A), Ins2^{Akita} mice treated with vehicle (FIG. 2B); control mice treated with arginase inhibitor (FIG. 2C), and Ins2^{Akita} mice treated with arginase inhibitor (FIG. 2D).

Vehicle-treated Ins2^{Akita} mice showed significant increases in glomerular macrophages (2.95±0.14 macrophages/glomerulus; p<0.0001) in FIG. 2B compared with vehicle-treated control (0.34±0.04 macrophages/glomerulus) in FIG. 2A. Both arginase inhibitor-treated control mice in FIG. 2C and Ins2^{Akita} mice in FIG. 2D showed significantly reduced glomerular macrophage recruitment (0.29±0.03 and 0.9±0.14 macrophages/glomerulus; p<0.0001); respectively compared to vehicle-treated Ins2^{Akita} mice; at 14 weeks of age.

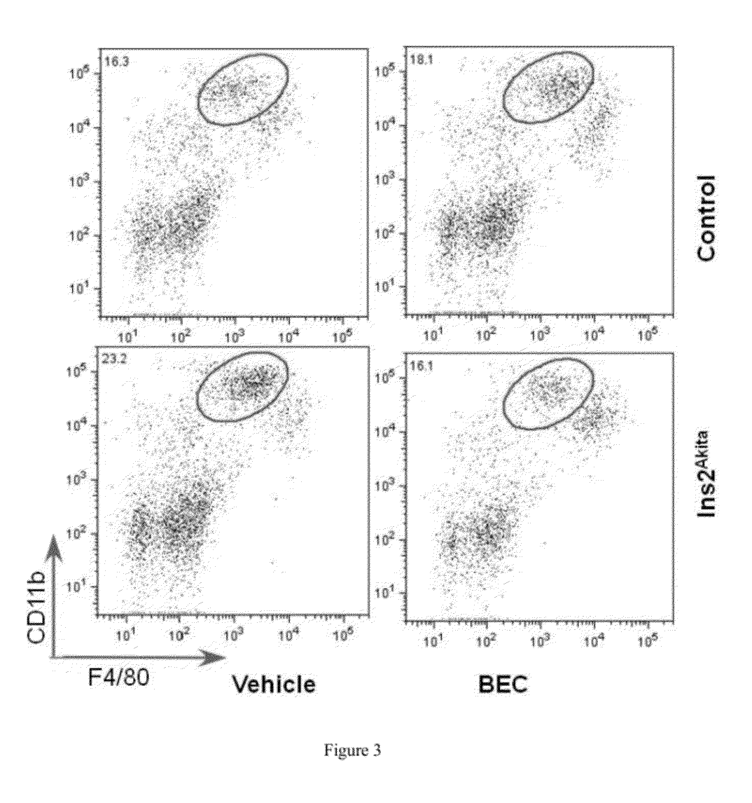

Similar results were obtained when kidney macrophages CD11b⁺F4/80^{low} were detected by FACS. FIG. 3 includes representative contour plots from kidneys harvested at 14 weeks of age and processed for FACS as described supra. FIG. 3 shows the effect of arginase inhibition on macrophage recruitment in Ins2^{Akita} mice using FACS.

Arginase 2 but not Arginase 1 is Expressed and Regulated in Ins2^{Akita} Mice

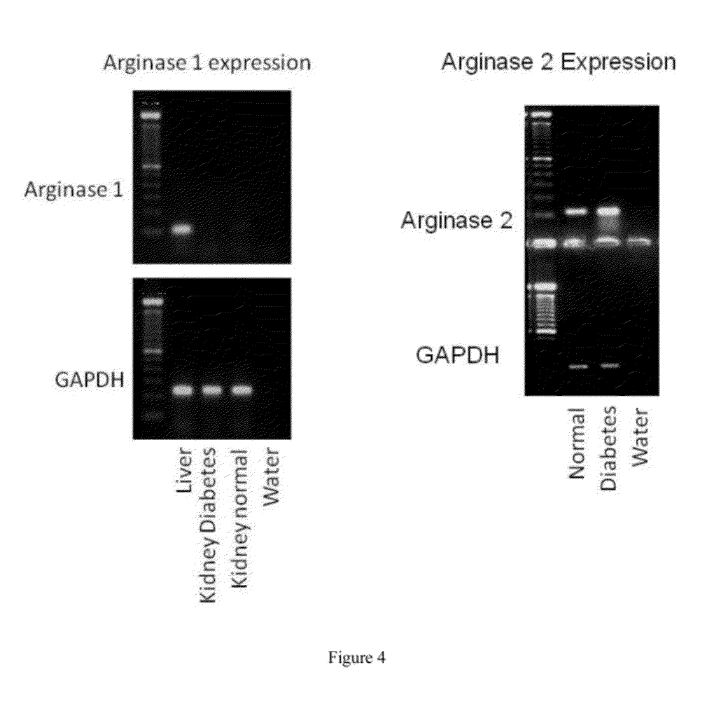

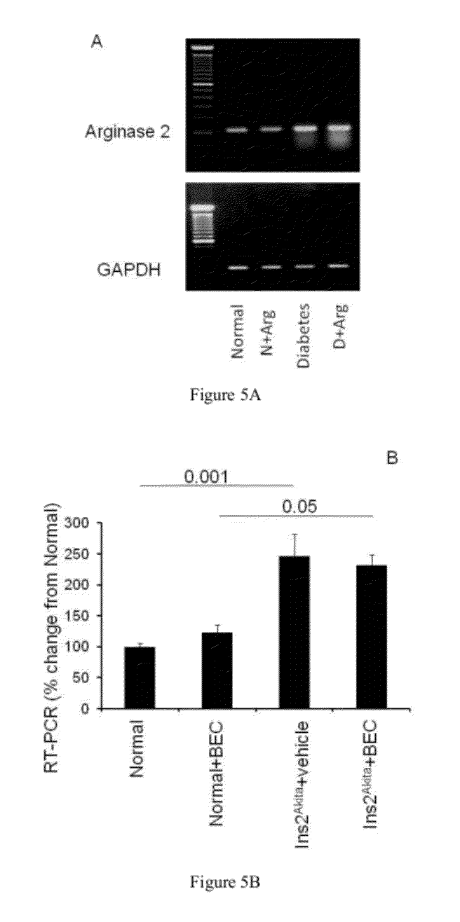

An assessment was conducted on whether both arginases (arginase 1 and arginase 2) were expressed on the kidneys under normal and diabetic conditions. As shown in FIG. 4, only arginase 2 was expressed in the kidney but not arginase 1. Therefore, the effect of arginase inhibitor is mainly mediated through kidney arginase 2 rather than arginase 1.

RT-PCR was performed on whole mouse kidney total RNA isolated at 14 weeks of age. FIG. 5A shows a gel analysis of PCR products. In FIG. 5B, expression of arginase 2 mRNA was normalized to GAPDH and data were calculated as expression relative to control. FIGS. 5A and 5B show that both vehicles treated and arginase inhibitor treated Ins2^{Akita} mice led to ˜1.5-fold increase in arginase 2 mRNA expressions. Thus, diabetes is associated with increased arginase 2 expression and that arginase inhibition does not down regulate arginase expression in diabetes.

Inhibition of Arginases Reduces Albuminuria in STZ-Induced Diabetic Mouse

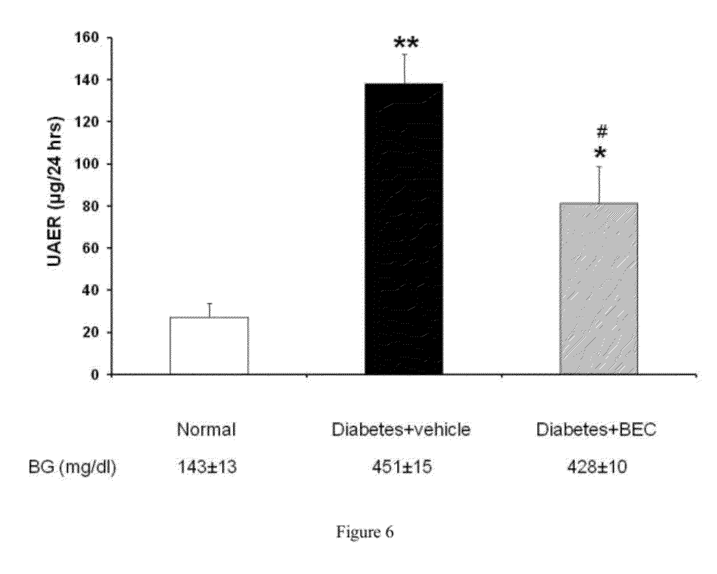

To determine if the effect of arginase inhibition is specific to the Ins2Akita mouse model, another type 1 diabetic mouse model (DBA background) was used, also using STZ to induce diabetes for a 6 weeks study period. 24-hr UAE was measured as indicators of renal injury with and without arginase inhibitor (BEC) treatment. As shown in FIG. 6, vehicle-treated STZ-induced diabetes had a significant increase in albuminuria compared to controls after 6 weeks, the effect significantly reduced with BEC treatment in diabetic mice despite comparable blood glucose levels.

An increase in kidney macrophages recruitment was also demonstrated using Mac-2 staining in vehicle-treated STZ-induced diabetic mice (1.9±0.1 macrophages/glomerulus; p<0.0001) compared with control mice (0.6±0.05 macrophages/glomerulus). In contrast, arginase inhibitor-treated STZ-induced diabetes significantly reduced glomerular macrophage recruitment (1.2±0.06 macrophages/glomerulus; p<0.0001) compared to vehicle-treated STZ-induced diabetic mice

Inhibition of Arginases Decreases Renal Histological Changes in STZ-Induced Diabetic Mice

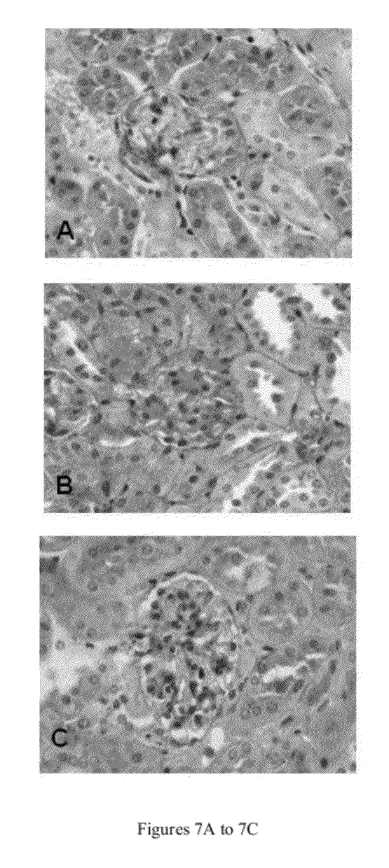

Periodic acid-Schiff (PAS) staining of kidney sections (as shown in FIGS. 7A to 7C) demonstrated increased glomerular cellularity and mesangial expansion (score: 1.9±0.05 vs. 1.1±0.09, p<0.005) after 6 weeks of diabetes in vehicle-treated STZ mice versus control mice, respectively. Inhibition of arginases in STZ-induced diabetic mice exhibited significantly reduced glomerular changes (scores: 1.6±0.08; p<0.05) compared to vehicle-treated STZ-induced mice.

Deficiency of Arginase 2 Reduces Albuminuria in Diabetic Mice

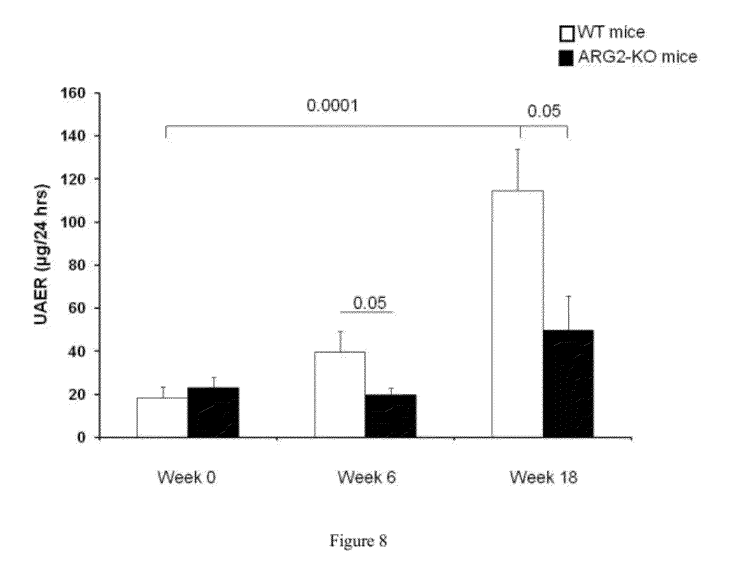

To assess whether lack of arginase 2 mimics the changes observed in Ins2^{Akita} mice after arginase inhibition, mice were treated with multiple low doses of STZ (50 mg/kg) for 5 days and euthanized at either 6 weeks or 18 weeks after STZ-induced diabetes in accordance with the above study protocol. Arginase 2^{+/+} mice displayed a significant increase in UAE after 6 weeks and after 18 weeks (see FIG. 8) of STZ-induced diabetes compared to non-diabetic control mice. In contrast, the increase in UAE was almost completely abrogated in mice lacking the arginase 2 gene in a similar manner to arginase inhibitor treatment in Ins2^{Akita}.

Deficiency of Arginase 2 Restores Renal Medullary Blood Flow in Diabetic Mice

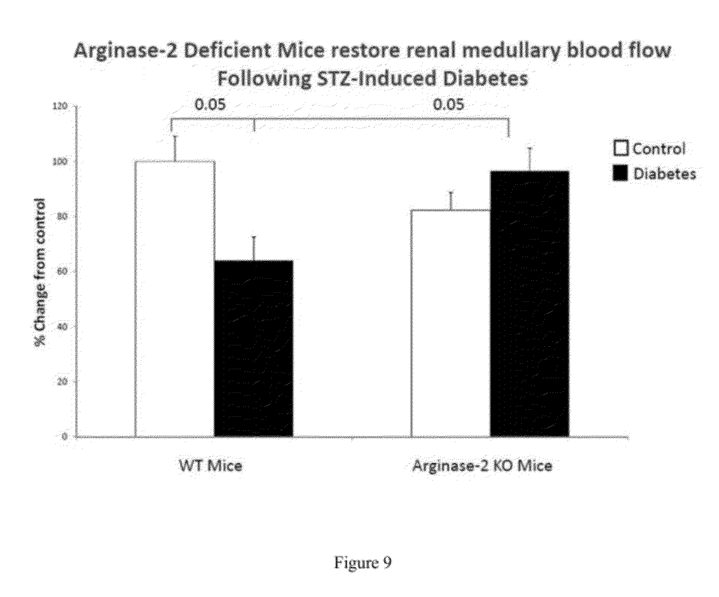

Both arginases and nitric oxide synthases compete for the same L-arginine substrate. Therefore, an assessment was conducted of whether lack of arginase 2 has an effect on renal blood flow. As shown in FIG. 9, diabetes was associated with reduced renal medullary blood flow—an effect completely reversed in arginase 2 deficient mice—indicating a possible role of nitric oxide action in arginase 2 deficient mice.

Increases of Kidney Arginase Activity in Diabetic Mice

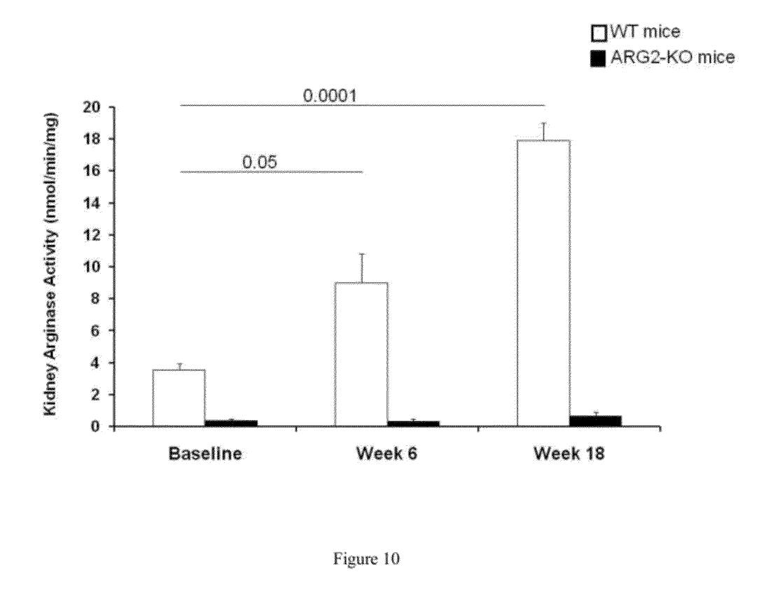

An assessment of kidney arginase activity in WT and ARG2 KO mice at baseline, week 6 and week 18 after STZ-induced diabetes was conducted. As shown in FIG. 10, diabetes was associated with a significant increase in kidney arginase activity at 6 weeks and 18 weeks after STZ-induced diabetes. In contrast, deficiency of arginase 2 in ARG2 KO mice displayed a very minimal kidney arginase activity at all time points indicating a little, if any, role for arginase 1 in the kidney under normal and diabetic conditions in ARG2 KO mice.

Arginase-2 Deficient Mice (Arg2^{-/-}) are not Hypertensive

Although Arg2^{-/-} mice have been reported to be hypertensive, blood pressure is not a cause of proteinuria in the Arg2^{-/-} mice. First, WT and Arg2^{-/-} mice had similar baseline levels of UAER, thus excluding any role of hypertension-induced albuminuria. Second, inhibition of arginases in adult spontaneously hypertensive rats decreased blood pressure. Third, an assessment of intra-carotid blood pressure in anesthetized WT and Arg2^{-/-} mice (6-9 wks of age) was conducted.

TABLE 2
Blood pressure measurements in Arg2^{+/+} and
Arg2^{-/-} mice (6-9 weeks of age):

| | Arg2^{+/+} | Arg2^{+/+} | P value |
|---|---|---|---|
| Number of mice | 5 | 6 | |
| SBP; mmHg | 107 ± 3 | 111 ± 2 | 0.4 |
| DBP; mmHg | 72 ± 1 | 79 ± 3 | 0.07 |
| Pulse Pressure | 36 ± 2 | 32 ± 1 | 0.2 |
| Heart Rate | 525 ± 18 | 519 ± 27 | 0.8 |

Data in Table 2 are mean±SEM. SBP: systolic blood pressure, DBP: diastolic blood pressure.

As shown in Table 2, there were no significant differences between WT and Arg2^{-/-} mice. As such, the results do not indicate that increased albuminuria is due to hypertension.

Effect of High Glucose on Arginase-2 Expression on Glomerular Endothelial Cells in Vitro

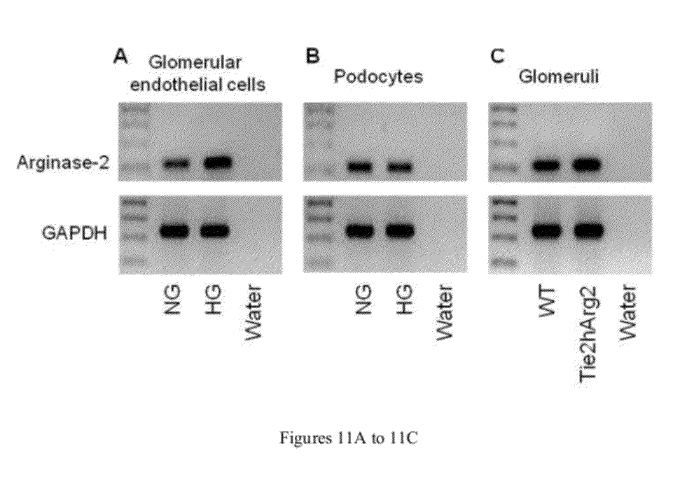

A previous study using cultured bovine endothelial cells found that high glucose (HG) induced arginase-1 activity by two-fold; strangely, but was no increase in the arginase-1 protein level. To assess species differences in expression of arginase isozymes, RT-PCR was performed on murine glomerular endothelial cells and podocytes supplemented with either normal glucose (NG) (11 mM) or HG (33 mM). These cells were differentiated for 10 days at 37° C. in DMEM/F12. Arginase-2 expression in the glomerular endothelial cells is shown in FIG. 11A. Arginase-2 expression in the podocytes is shown in FIG. 11B. Kidney glomeruli were isolated using magnetic beads from WT and Tie2hArg2 mice and were subjected to RT-PCR for arginase-2 expression (see FIG. 11C). GAPDH was used as a control gene.

As shown in FIG. 11A, HG media increased the arginase-2 mRNA level in glomerular endothelial cells by 158±15% (p<0.005) compared to NG. In contrast to the results with bovine cells, arginase-1 was not detectable. This data suggest that elevated arginase-2 expression may affect function of glomerular endothelial cells.

Localization and Expression of Arginase-2 in Mouse Kidney

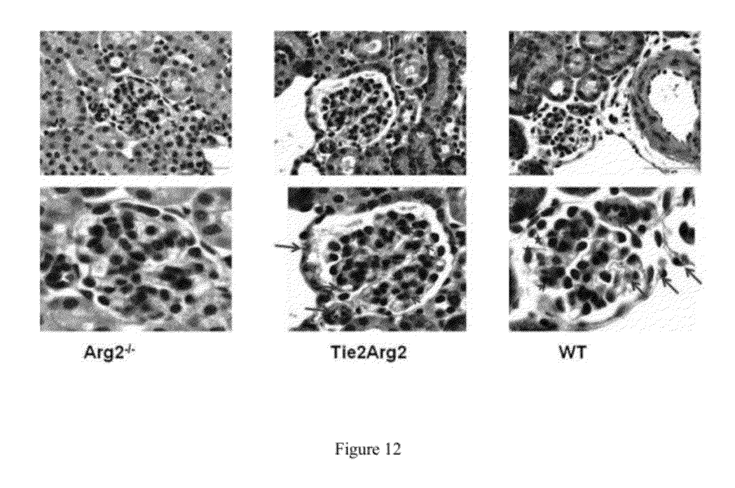

Immuno-histochemical staining for arginase-2 on representative kidney section was conducted. All sections were stained on a single slide to eliminate slide to slide variation. FIG. 12 shows the images and includes long arrows for endothelial cells, short arrows for mesangial cells, and stars for parietal and visceral podocytes.

As shown in FIG. 12, immunohistochemical studies that identified arginase-2 expression in proximal straight tubules and inner medullary collecting duct are confirmed. Arginase-2 expression was also identified in podocytes, isolated glomeruli, and mesangial cells. Furthermore, both Tie2hArg2 transgenic and WT mice showed strong granular cytoplasmic (mitochondrial) staining for arginase-2 in tubular epithelial cells, with specific staining within endothelial cells, mesangial cells, and visceral podocytes. The Arg2^{-/-} mouse had no specific staining, confirming specificity of the immunohistochemistry. The results suggest a role for arginase-2 in these kidney cell types.

Arginase-2 is Expressed in Human Diabetic Kidneys

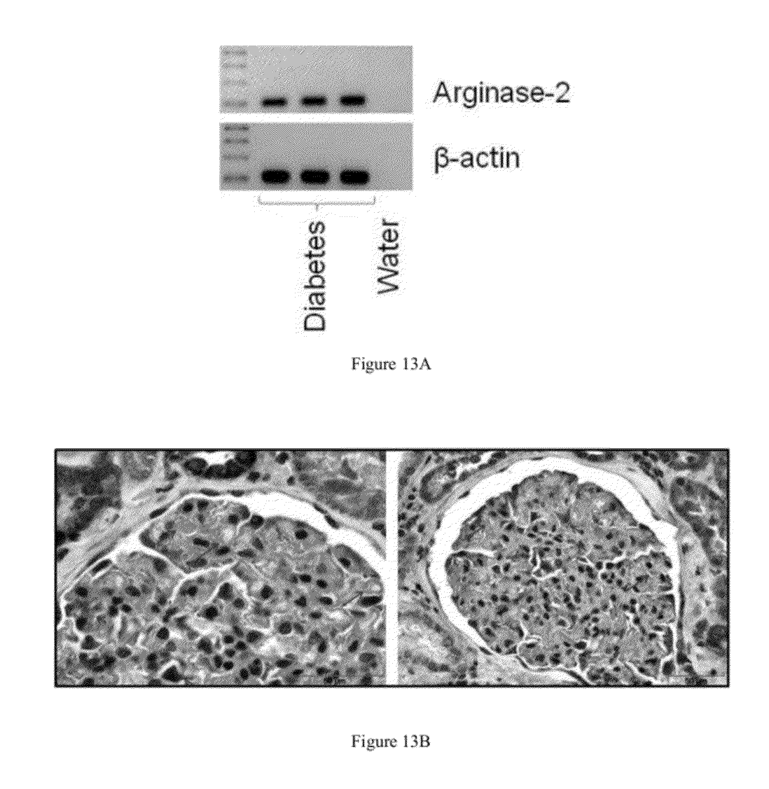

An initial RT-PCR and immunohistochemistry of human diabetic kidney samples (type-2 diabetes) was conducted and demonstrated expression of arginase-2, as shown in FIG. 13A. Immunohistochemical staining shown in FIG. 13B includes long arrows for endothelial cells, short arrows for mesangial cells, and stars for podocytes. FIG. 13B shows granular cytoplasmic (mitochondrial) staining in tubular epithelial cells, endothelial, mesangial cells and podocytes.

Renal Tissue-Protective Effect of Arginase Inhibition is Endothelial Nitric Oxide Synthase (eNOS) Dependent Following Diabetes

Preliminary data showed that deficiency of arginase-2 restores renal medullary blood flow in diabetic mice, possibly through an effect on nitric oxide. Because arginases and nitric oxide synthases can compete for L-arginine, an assessment was conducted of whether the effect of arginase inhibition in diabetic nephropathy is eNOS dependent.

eNOS deficient mice are recommended by the Animal Models of Diabetes Complications Consortium (AMDCC) as an optimal model of diabetic nephropathy. Experiments were conducted in male 6-week-old eNOS deficient and their wild type (WT) littermate mice using multiple low doses of STZ (50 mg/kg body weight for 5 days). Arginase inhibition using BEC (2.3 mg/kg/day) or vehicle was administered by continuous subcutaneous infusion for 6 weeks via a mini-osmotic pump. Renal function was monitored throughout the study.

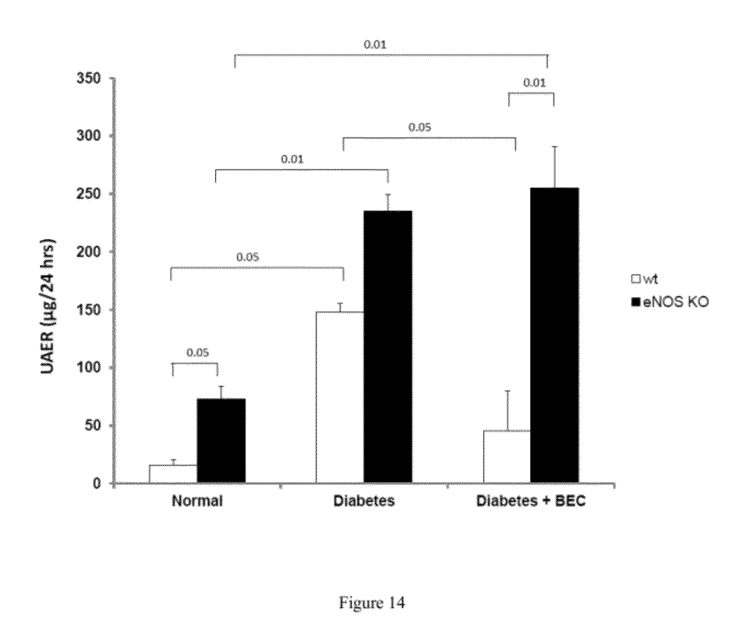

As shown in FIG. 14, normal eNOS deficient mice exhibited increases in urinary albumin excretion (UAE) than WT mice due to a reduction in nitric oxide and increases in blood pressure. Whereas vehicle-treated WT mice had a significant increase UAE compared to controls after 6 following diabetes, albuminuria was significantly reduced in diabetic WT mice treated with BEC. In contrast, BEC treated eNOS deficient mice failed to reduce UAE in diabetic eNOS deficient mice compared to vehicle treated diabetic mice. This data indicated that the effect of arginase inhibition, at least, is mediated via a nitric oxide dependent mechanism.

### CONCLUSION

Pharmacological blockade or genetic deficiency of arginase-2 confers kidney protection in Ins2^{Akita} or STZ-induced diabetic mice kidney disease. Blocking arginases using BEC for 9 weeks in Ins2^{Akita} mice or 6 weeks in STZ-induced diabetic model (DBA background) significantly attenuated albuminuria, the increase in blood urea nitrogen, histological changes and kidney macrophage recruitment compared to vehicle. Furthermore, kidney arginase-2 expressions increased in Ins2^{Akita} mice compared to control. In contrast, arginase-1 expression was undetectable in the kidneys under normal or diabetic conditions. Lack of arginase-2 (arg-2^{-/-}) in mice mimicked arginases blockade by reducing albuminuria to normal range after 6 weeks (p<0.05) and after 18 weeks (p<0.05) of STZ-induced diabetes compared to arg-2^{+/+} mice despite comparable blood glucose levels. In arg-2^{+/+} mice, kidney arginase activity increased significantly after 6 weeks (p<0.05) and after 18 weeks (p<0.0001) of STZ-induced diabetes while it was very low in arg-2^{-/-}. The increase in kidney arginase activity was associated with a reduction in renal medullary blood flow (p<0.05 compared to control) in arg-2^{+/+} mice after 6 weeks of STZ-induced diabetes, an effect significantly reduced (p<0.05) in diabetic arg-2^{-/-} mice.

Accordingly, the methods of the invention as described herein are a promising approach in the treatment of diabetic renal injury, providing a beneficial effect on diabetic nephropathy and ameliorating diabetic renal injury.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A method of treating renal injury comprising:
administering to a subject in need thereof, a composition comprising a therapeutically effective amount of at least one of: an arginase inhibitor, an L-citrulline, an L-citrulline supplement or combinations thereof; and
treating renal injury.

2. The method of claim 1, wherein the composition comprising a therapeutically effective amount of at least one of: the arginase inhibitor the L-citrulline, the L-citrulline supplement or combinations thereof, is administered together with a pharmaceutically acceptable carrier.

3. The method of claim 1, wherein the composition is administered by intravenous injection, orally, intraperitoneal injection, sub-cutaneous injection, continuous infusion or combinations thereof.

4. The method of claim 1, wherein the subject comprises a human.

5. The method of claim 1, wherein the renal injury comprises diabetic nephropathy.

6. The method of claim 1, wherein the renal injury comprises albuminuria.

7. A method of inhibiting arginase in vivo comprising:
administering in vivo to a subject in need thereof, a therapeutically effective amount of an arginase inhibitor;
wherein inhibition of arginase treats renal injury.

8. The method of claim 7, wherein the subject comprises a human.

9. The method of claim 7, wherein the arginase inhibitor selectively inhibits arginase 2 (ARG2), or arginase 1 (ARG1) or both arginase 2 and 1.

10. The method of claim 7, wherein the arginase inhibitor comprises an oligonucleotide specific for ARG2 or ARG1 or combinations thereof.

11. The method of claim 7, wherein the arginase inhibitor comprises an antibody specific for an arginase 2 enzyme, arginase 1 enzymes or combinations thereof.

12. The method of claim 7, wherein the arginase inhibitor comprises an L-arginine supplement or L-Citrulline supplement.

13. The method of claim 12, wherein the L-arginine supplement comprises natural or synthetic L-arginine, variants, analogs, derivatives, enantiomers, substitutions, fragments or combinations thereof.

14. The method of claim 12, wherein the L-Citrulline supplement comprises natural or synthetic L-Citrulline, variants, analogs, derivatives, enantiomers, substitutions, fragments or combinations thereof.

15. The method of claim 1, wherein the arginase inhibitor is administered together with a pharmaceutically acceptable carrier.

16. A method for identifying an agent for modulating arginase in vitro or in vivo comprising:
providing a test substance;
determining whether the test substance is an arginase inhibitor; and
selecting the test substance for treatment of modulating arginase if the test substance is an arginase inhibitor.

17. The method of claim 16, further comprising:
determining whether the test substance selectively inhibits arginase; and wherein the selecting step further comprises selecting the test substance for treatment of diabetic renal injury if the test substance is an arginase inhibitor and selectively inhibits arginase 2 or arginase 1 or combinations thereof.

18. The method of claim 16, wherein the arginase inhibitor inhibits arginase activity.

19. The method of claim 16, wherein the arginase inhibitor increases nitric oxide production, function, bioavailability or activity.
